(19) United States
(12) Patent Application Publication
(10) Pub. No.: US 2007/0178093 A1
(43) Pub. Date: Aug. 2, 2007

(54) LYMPHOCYTES; METHODS

(75) Inventors: Shino Hanabuchi, Houston, TX (US); Rene de Waal Malefyt, Sunnyvale, CA (US); Yong-Jun Liu, Pearland, TX (US)

Correspondence Address: DNAX RESEARCH INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 11/675,190

(22) Filed: Feb. 15, 2007

Related U.S. Application Data
(63) Continuation of application No. 10/888,437, filed on Jul. 8, 2004, now abandoned.
(60) Provisional application No. 60/486,621, filed on Jul. 11, 2003.

(51) Int. Cl. A61K 39/395

(52) U.S. Cl. 424/144.1

(57) ABSTRACT

Provided are methods of modulating activity of regulatory T cells, CD4⁺ T cells, and CD8⁺ T cells. Also provided are methods of treating immune disorders.

This application is a Continuation of U.S. patent application Ser. No. 10/888,437, filed Jul. 8, 2004, which claims benefit of U.S. Provisional Patent Application No. 60/486,621, filed Jul. 11, 2003, each of which is incorporated herein by reference.

### FIELD OF THE INVENTION

The invention provides methods of modulating the physiology of cells, e.g., dendritic cells, regulatory T cells, and naïve T cells. Also provided are methods of modulating immune disorders, e.g., inflammatory and proliferative disorders.

### BACKGROUND

Cancer, persistent infections, and old age pose unusual problems to the immune system (Ferenczy and Franco (2002) Lancet Oncol. 3:11-16; Vyas (2000) Dev. Biol. Stand. 102:9-17; Saurwein-Teissl, et al. (2002) J. Immunol. 168:5893-5899; Melby (2002) Am. J. Clin. Dermatol. 3:557-570; Pardoll (2003) Ann. Rev. Immunol. 21:807-839). Infections and cancers, for example, can persist because of overactivity of regulatory T cells (a.k.a. Tr cells; Treg cells; reg T cells; Tregs). A number of Treg cells have been identified, e.g., CD25⁺CD4⁺ T cells, Th3 cells, and Tr1 cells. Overactivity of these Treg cells can contribute to the resistance of tumors and infections to the immune system, where this resistance may take the form of, e.g., tolerance to the tumor, progressing lesions in cancer, and persistent bacterial and viral infections, see, e.g., Shimizu, et al. (2002) Nat. Immunol. 3:135-142; Shimizu, et al. (1999) J. Immunol. 163:5211-5218; Antony and Restifo (2002) J. Immunotherapy 25:202-206; McGuirk and Mills (2002) Trends Immunol. 23:450-455; Tatsumi, et al. (2002) J. Exp. Med. 196:619-628; Jonuleit, et al. (2001) Trends Immunol. 22:394-400.

Additionally, Treg cells mediate inflammatory and autoimmune disorders. For example, CD25⁺CD4⁺ Treg cells play a role in preventing, e.g., autoimmune gastritis, thyroiditis, insulin-dependent diabetes melitus (IDDM), inflammatory bowel disorders (IBD), experimental autoimmune encephalomyelitis (EAE), food allergies, and graft rejection. Conversely, impaired Treg cell activity can promote autoimmune disorders, see, e.g., Wing, et al. (2003) Eur. J. Immunol. 33:579-587; Sakaguchi, et al. (2001) Immunol. Revs. 182:18-32; Suri-Payer, et al. (1998) J. Immunol. 160:1212-1218; Shevach (2001) J. Exp. Med. 193:F41-F45; Read and Powrie (2001) Curr. Op. Immunol. 13:644-649.

Furthermore, Treg cells have been implicated in neuroprotection. Injury to the nervous system, e.g., spinal trauma, can result in infiltration of lymphocytes at the site of injury, followed by pathological nerve damage, e.g., involving neuronal death. This damage can be prevented by Treg cells (Yoles, et al. (2001) J. Neuroscience 21:3740-3748; Jones, et al. (2002) J. Neuroscience 22:2690-2700).

Treg cells can suppress activity and proliferation of CD8⁺ T cells and CD4⁺ T cells. CD8⁺ T cells contribute to the pathology of inflammatory disorders such as psoriasis and other skin conditions, rheumatoid arthritis, and IBD, see, e.g., Liblau, et al. (2002) Immunity 17:1-6; Deguchi, et al. (2001) Arch. Dermatol. Res. 293:442-447; Sigmundsdottir, et al. (2001) Clin. Exp. Immunol. 126:365-369; Kang, et al. (2002) J. Exp. Med. 195:1325-1336; Muller, et al. (1998) Am. J. Pathol. 152:261-268; Homma, et al. (2001) Hepatogastroenterol. 48:1604-1610. CD4⁺ T cells contribute to the pathology of asthma and allergies, systemic lupus erythematosus, rheumatoid arthritis, and psoriasis, see, e.g., Cope (2002) Arthritis Res. 4 Suppl. 3:S197-211; Prinz (1999) Exp. Dermatol. 24:291-295; Sugimoto, et al. (2002) Autoimmunity 35:381-387; Tattersfield, et al. (2002) Lancet 360:1313-1322. Moreover, CD4⁺ and CD8⁺ T cells are used for combating infections and pathological proliferative conditions, e.g., cancer and tumors, see, e.g., Titu, et al. (2002) Cancer Immunol. Immunother. 51:235-247; Ho, et al. (2002) J. Clin. Invest. 110:1415-1417; Wong and Pamer (2003) Annu. Rev. Immunol. 21:29-70. A number of functional differences between mouse and human CD8⁺ T cells have been described, see, e.g., McAdam, et al. (2000) J. Immunol. 165:3088-3093; Kreisel, et al. (2002) J. Immunol. 169:6154-6161; Hamann, et al. (1997) J. Exp. Med. 186:1407-1418.

There is an unmet need to treat infections and cancers that do not respond to the normal immune system, as well as a need to treat inflammatory and autoimmune disorders. This invention addresses these needs by providing methods to break the suppressive effects of regulatory T cells, methods to modulate the activity of CD8⁺ T cells, and methods to prepare mature dendritic type 2-cells.

### SUMMARY OF THE INVENTION

The present invention is based, in part, upon the discovery that TEASR and TEASR-L activity can modulate cell proliferation.

The present invention provides a method of modulating proliferation of a human cell comprising contacting the cell with an agonist of glucocorticoid-induced tumor necrosis factor family-related receptor (TEASR) or of TEASR-L ligand (TEASR-L); or an antagonist of TEASR or of TEASR-L. Also provided is this method wherein the agonist increases cell proliferation; or wherein the antagonist decreases cell proliferation; or the above method wherein the cell is a human CD8⁺ T cell; or the above method wherein the agonist or antagonist is a binding composition that specifically binds to TEASR or to TEASR-L; or the above method wherein the binding composition is derived from the antigen binding site of an anti-TEASR antibody or an anti-TEASR-L antibody; or the above method wherein the binding composition is a polyclonal antibody; a monoclonal antibody; a human antibody or a humanized antibody; an Fab or F(ab′)₂ fragment; a peptide mimetic of an antibody; a soluble TEASR or soluble TEASR-L; or detectably labeled.

Yet another aspect of the present invention provides a method of treating a human immune disorder comprising treatment or administration with an antagonist of TEASR; or this method wherein the immune disorder is psoriasis; rheumatoid arthritis; an inflammatory bowel disorder (IBD); or a CD8⁺ T cell-mediated disorder; or the above method wherein the antagonist of TEASR is a binding composition that specifically binds to TEASR-L; as well as the above method wherein the binding composition is a polyclonal antibody; a monoclonal antibody; a human antibody or a humanized antibody; an Fab or F(ab′)₂ fragment; a peptide mimetic of an antibody; a soluble TEASR; or detectably labeled.

Provided is a method of treating a human proliferative disorder comprising treatment or administration with an agonist of TEASR; the above method wherein the agonist comprises a binding composition that specifically binds to TEASR; and the above method wherein the binding composition is a polyclonal antibody; a monoclonal antibody; a human antibody or a humanized antibody; an Fab or F(ab′)2 fragment; a peptide mimetic of an antibody; a soluble TEASR-L; or detectably labeled.

### DETAILED DESCRIPTION

As used herein, including the appended claims, the singular forms of words such as “a,” “an,” and “the,” include their corresponding plural references unless the context clearly dictates otherwise. All references cited herein are incorporated by reference to the same extent as if each individual publication, patent application, or patent, was specifically and individually indicated to be incorporated by reference.

I. Definitions.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. Treatment encompasses methods using a purified immune cell, e.g., in a mixed cell reactions or for administration to a research, animal, or human subject. The invention contemplates treatment with a cell, a purified cell, a stimulated cell, a cell population enriched in a particular cell, and a purified cell. Treatment further encompasses situations where an administered reagent or cell is modified by metabolism, degradation, or by conditions of storage.

“Allogeneic,” as it applies to cells or to a reaction between cells, refers, e.g., to an interaction where the major histocompatibility complex (MHC) of a first cell is recognized as foreign by a second cell. “Autologous,” as it applies to cells or to a reaction between cells, refers, e.g., to an interaction where the MHC of a first cell is recognized as self by a second cell (Abbas, et al. (2000) Cellular and Molecular Immunology, 4^{th} ed., W.B. Saunders Co., Philadelphia).

“Conservatively modified variants” applies to both amino acid and nucleic acid sequences. With respect to particular nucleic acid sequences, conservatively modified variant refers to those nucleic acids that encode identical or essentially identical amino acid sequences. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132):

(1) Hydrophobic: Norleucine, Ile, Val, Leu, Phe, Cys, Met;

(2) Neutral hydrophilic: Cys, Ser, Thr;

(3) Acidic: Asp, Glu;

(4) Basic: Asn, Gln, His, Lys, Arg;

(5) Residues that influence chain orientation: Gly, Pro;

(6) Aromatic: Trp, Tyr, Phe; and

(7) Small amino acids: Gly, Ala, Ser.

“Effective amount” means an amount sufficient to ameliorate or prevent a symptom or sign of the medical condition.

“Exogenous” refers to substances that are produced outside an organism, cell, or human body, depending on the context. “Endogenous” refers to substances that are produced within a cell, organism, or human body, depending on the context.

An “immunoassay” is an assay that uses an antibody, or antigen-binding fragment thereof, to specifically bind an antigen. The immunoassay is characterized by the use of specific binding properties of a particular antibody to isolate, target, detect, or quantify the antigen.

“Immunosuppression” refers to the reduction, depression, or damping of immune response. Immunosuppression includes tolerance, e.g., antigen-specific tolerance (Delves and Roitt (eds.) (1998) Encyclopedia of Immunology, Academic Press, Inc., San Diego, Calif.). Immunosuppression may be a normal or pathological phenomenon, or may result from an underlying disorder or from an immunosuppressive drug or pharmacological agent.

“Inhibitors” and “antagonists” or “activators” and “agonists” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis, see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126.

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Purified” and “enriched” means that the concentration or specific activity of, e.g., a molecule, complex, or cell, is greater than that found in a parent sample or greater than that of a predetermined standard sample.

“Nucleic acid” refers to deoxyribonucleotides or ribonucleotides and polymers thereof in either single stranded or double-stranded form. The term nucleic acid may be used interchangeably with gene, cDNA, mRNA, oligonucleotide, and polynucleotide. A particular nucleic acid sequence also implicitly encompasses “allelic variants” and “splice variants.” Splice variants of TEASR have been identified, e.g., see Nocentini, et al. (2000) Cell Death and Differentiation 7:408-410.

“Soluble receptor” refers to receptors that are water-soluble and occur, e.g., in extracellular fluids, intracellular fluids, or weakly associated with a membrane. Soluble receptor also refers to receptors that have been released from tight association with a membrane, e.g., by limited proteolytic cleavage or cleavage of a lipid that maintains binding of the receptor to the membrane. Furthermore, soluble receptor encompasses receptors that are biochemically or chemically modified or engineered to be water soluble.

The invention contemplates use of a soluble TEASR and a soluble TEASR-L, as well as fragments thereof that are capable of binding to a ligand or receptor. Also contemplated are soluble receptors comprising an Ig fusion protein, see, e.g., Harris, et al. (2002) J. Immunol. Methods 268:245-258; Corcoran, et al. (1998) Eur. Cytokine Netw. 9:255-262; Mackay, et al. (1997) Eur. J. Immunol. 27:2033-2042. Soluble TEASRs and soluble TEASR-Ls have been identified, see, e.g., Nocentini, et al. (2000) Cell Death and Differentiation 7:408-410; Gurney, et al (1999) Curr. Biol. 9:215-218; Shin, et al. (2002) FEBS Letters 514:275-280. Ig fusion protein ligands may contain a mutation (D265A in the constant regions of the Fc) to prevent binding to Fc receptor (FcR) and to complement (Idusogie, et al. (2000) J. Immunol. 164:4178-4184). General methods relating to soluble receptors have been described, see, e.g., Monahan, et al. (1997) J. Immunol. 159:4024-4034; Moreland, et al. (1997) New Engl. J. Med. 337:141-147; Borish, et al. (1999) Am. J. Respir. Crit. Care Med. 160:1816-1823; Uchibayashi, et al. (1989) J. Immunol. 142:3901-3908.

“Specifically” or “selectively” binds, when referring to a ligand/receptor, antibody/antigen, or other binding pair, e.g., TEASR-L to TEASR, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the antibody, or binding composition derived from the antigen-binding site of an antibody, of the contemplated method binds to its antigen, or a variant or mutein thereof, with an affinity that is about two fold greater, preferably ten times greater, more preferably 20-times greater, and most preferably 100-times greater than the affinity with any other antibody, or binding composition derived thereof. In a preferred embodiment the antibody will have an affinity which is greater than about 10⁹ liters/mol, as determined, e.g., by Scatchard analysis (Munsen, et al. (1980) Analyt. Biochem. 107:220-239).

“Ligand” refers to small molecules, peptides, polypeptides, and membrane associated or membrane-bound molecules that act as agonists or antagonists of a receptor, to agents that maintain binding that are not agonists or antagonists, as well as to soluble versions of ligands that are membrane-associated or membrane-bound. By convention, where a ligand is membrane-bound on a first cell, the receptor usually occurs on a second cell. The second cell may have the same or a different identity as the first cell. A ligand or receptor may be entirely intracellular, that is, it may reside in the cytosol, nucleus, or some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist irradication by the immune system. “Cancerous condition” includes, e.g., cancer, cancer cells, tumors, angiogenesis, and precancerous conditions such as dysplasia.

“Sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

“Therapeutically effective amount” of a therapeutic agent is defined as an amount of each active component of the pharmaceutical formulation that is sufficient to show a meaningful patient benefit, i.e., to cause a decrease in, prevention, or amelioration of the symptoms of the condition being treated. When the pharmaceutical formulation comprises a diagnostic agent, “a therapeutically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual, see, e.g., U.S. Pat. No. 5,888,530.

II. Glucocorticoid-Induced Tumor Necrosis Factor Family-Related Receptor (TEASR).

The invention contemplates methods of modulating the activity of TEASR and/or TEASR-L, as well as methods of modulating activity of cells expressing TEASR and/or TEASR-L. Human TEASR-L is also known as AITRL, DNA19355, and GLITTER. TEASR-L, TEASR, and their variants, have been described, see, e.g., Gurney, et al. (1999) Current Biol. 9:215-218; Nocentini, et al. (2000) Cell Death Differ. 7:408-410; Kwon, et al. (1999) J. Biol. Chem. 274:6056-6061; Kwon, et al. (2003) Exp. Mol. Med. 35:8-16; SEQ ID NO:2 of WO 98/07880; GenBank NM_{—}005092. Expression of TEASR-L and TEASR have been described for human and mouse cells and tissues, see, e.g., Shimizu, et al. (2002) supra; Gurney, et al., supra; Kwon, et al. (1999) J. Biol. Chem. 274:6056-6061; Shin, et al. (2002) Cytokine 19:187-192; Shin, et al. (2002) FEBS Lett. 514:275-280; U.S. Pat. Pub. No. US 2002/0146389.

Tolerance is mediated, in part, by glucocorticoid-induced tumor necrosis factor family-related receptor (TEASR) (a.k.a. GITR; TNFRSF18; 312C2) and its ligand, TEASR-L (a.k.a. GITRL; TNFSF18). Self-tolerance can be accomplished by, e.g., clonal deletion, anergy, and by T regulatory cells (Tregs) (Roncarolo, et al. (2001) Immunol. Revs. 182:68-79).

TEASR modulates autoimmune disorders, as shown by work on depleting TEASR-expressing cells or by treating animals with cells that express TEASR. Depletion of TEASR-expressing T cells results in autoimmune disorders, e.g., gastritis and inflammation of the ovaries (Shimizu, et al. (2002) Nature Immunol. 3:135-142).

TEASR is a signaling molecule, as shown by studies using TEASR-L or activating anti-TEASR antibodies to stimulate TEASR (Gurney, et al., supra; Shimizu, et al., supra.

A connection between TEASR mediated signaling and Treg cell activity was shown. Mouse CD25⁺CD4⁺ T cells suppressed proliferation of the CD25⁻CD4⁺ T cells in absence of antibody, where anti-TEASR antibody provided relief from this suppression, resulting in enhanced proliferation of the CD25⁻CD4⁺ T cells (Shimizu, et al.(2002) supra; McHugh, et al. (2002) Immunity 16:311-323).

As TEASR can be expressed by Treg cells, as well as by CD4⁺ T cells, studies have addressed the question of whether anti-TEASR antibody stimulated proliferation by breaking the suppressive effect of CD25⁺CD4⁺ Treg cells, by directly stimulating the CD25⁻CD4⁺ T cells to proliferate, or by both of these mechanisms. Anti-TEASR antibody was found to mediate T cell proliferation by both of these mechanisms (Shimizu, et al. (2002) supra).

CD25⁻CD4⁺ T cells can also mediate suppression under specific conditions, e.g., where the source of cells is aged mice. CD25⁻CD4⁺ T cells from aged mice can mediate suppression. In short, CD25⁻CD4⁺ T cells from aged mice can inhibit proliferation of co-cultured CD25⁻CD4⁺ T cells from young mice. The suppressive effect of the CD25⁻CD4⁺ T cells from aged mice is enhanced by activating these cells, e.g., with anti-CD3. Anti-TEASR antibody abrogates or breaks the suppressive effect of the CD25⁻CD4⁺ T cells from aged mice (Shimizu and Moriizumi (2003) J. Immunol. 170:1675-1682).

III. Regulatory T Cells.

Tregs of human origin include CD4⁺CD25+Tr cells, CD8⁺ Tr cells, NKT cells, Tr1 cells, Th3 cells, and CD8+ CD28- T cells. The terms “regulatory CD25⁺CD4⁺ T cell,” “CD25⁺CD4⁺ T cell,” “CD25⁺CD4⁺ Tr cell,” and “CD25⁺CD4⁺ Treg cell” refer to the same type of cell.

Human CD4⁺CD25+Treg cell-mediated suppression of CD4⁺CD25- T cell proliferation can be a function of the state of activation of the TCR, see, e.g., Baecher-Allan, et al. (2002) J. Immunol. 169:6210-6217; Shevach (2001) J. Exp. Med. 193:F41-F45; Levings, et al. (2001) J. Exp. Med. 193:1295-1302; Dieckmann, et al. (2001) J. Exp. Med. 193:1303-1310; Jonuleit, et al. (2001) J. Exp. Med. 193:1285-1294; Stephens, et al. (2001) Eur. J. Immunol. 31:1247-1254; Taams, et al. (2001) Eur. J. Immunol. 31:1122-1131; Baecher-Allan, et al. (2001) J. Immunol. 167:1245-1253.

Human CD8⁺ Tregs have been described (Gilliet and Liu (2002) J. Exp. Med. 195:695-704; Cortesini, et al. (2001) Immunol. Rev. 182:201-206; Colovai, et al. (2003) Hum. Immunol. 64:31-37; Saurwein-Teissl, et al. (2002) J. Immunol. 168:5893-5899; Horiuchi, et al. (2001) Bone Marrow Transplantation 27:731-739).

Human natural killer T cells (NKT cells) are comprised of a number of subsets, where one of these subsets has been identified as a Treg cell, see, e.g., Kadowaki, et al. (2001) J. Exp. Med. 193:1221-1226; Read and Powrie (2001) Curr. Op. Immunol. 13:644-649; Wang, et al. (2001) J. Exp. Med. 194:313-319; Godfrey, et al (2000) Immunol. Today 21:573-583.

Treg cells have also been identified in rodents, see, e.g., Gilliet and Liu (2002) Human Immunol. 63:1149-1155; MacDonald (2002) Gut 51:311-312; Caddle, et al. (1994) Immunity 1:553-562; Annacker, et al. (2001) J. Immunol. 166:3008-3018; Lehuen, et al. (1998) J. Exp. Med. 188:1831-1839; Bach (2001) Scand. J. Immunol. 54:21-29; Sakaguchi, et al. (1985) J. Exp. Med. 161:72-87; Schwartz and Kipnis (2002) Trends Immunol. 32:530-534; Nakamura, et al. (2001) J. Exp. Med. 194:629-644; Read and Powrie (2001) Curr. Op. Immunol. 13:644-649; Stephens and Mason (2000) J. Immunol. 165:3105-3110; Asseman, et al. (1999) J. Exp. Med. 190:995-1003; Davies, et al. (1999) J. Immunol. 163:5353-5357; Zuany-Amorim, et al. (2002) Nature Med. 8:625-629. Mouse CD25⁺CD4⁺ T cells may require activation to acquire suppressive activity, e.g., with anti-CD3 and IL-2 (McHugh, et al., supra).

IV. Dendritic Cells.

Dendritic cells are the most potent type of antigen-presenting cell (APC). DCs can induce self-tolerance, as well as the activation, polarization, and proliferation of T cells. The term “DC” is used herein to refer to immature, mature, inactivated, and activated DCs.

Three major subsets of human DC precursors have been identified:

(1) CD4⁺CD11c⁺CD14⁺ precursors (myeloid) (a.k.a. pre-DC1);

(2) CD4⁻CD11c⁺CD14⁻ immature myeloid; and

(3) CD4⁻CD11c⁻IL-3Ralpha⁺ precursors (lymphoid) (a.k.a. pre-DC2) (Nestle (2000) Oncogene 19:6673-6679; Woltman and van Kooten (2003) J. Leukocyte Biol. 73:428-441; O'Keefe, et al. (2003) Blood 101:1453-1459; Jonuleit, et al. (2001) Trends Immunol. 22:394-400; Damiani, et al. (2002) Bone Marrow Transpl. 30:261-266; Arpinati, et al. (2000) Blood 95:2484-2490).

Human CD4⁺CD11c⁻ plasmacytoid pre-DC2 (DC2 precursors) treated with IL-3 differentiate to immature DC2. In contrast to the situation with pre-DC1 monocytes, IL-4 can kill plasmacytoid pre-DC2 cells. Pre-DC2 treated with CD40 ligand or CpG motif nucleic acids differentiate to mature DC2. Mature DC2 can stimulate cell proliferation and cell differentiation, as follows. Mature DC2 can stimulate naïve CD4⁺ and CD8⁺ T cells to proliferate. Mature DC2 can stimulate naïve CD8⁺ T cells to differentiate, see, e.g., Liu (2002) Human Immunol. 63:1067-1071; Kadowaki, et al. (2001) J. Immunol. 166:2291-2295; Grouard, et al. (1997) J. Exp. Med. 185:1101-1111; Kadowaki, et al. (2001) J. Exp. Med. 194:863-869; Shortman and Liu (2002) Nature Revs. Immunol. 2:151-161; Liu (2002) Human Immunol. 63:1067-1071; Siegal, et al. (1999) Science 284:1835-1837; Guermonprez, et al., supra; Fong and Engleman, supra; Rissoan, et al. (1999) Science 283:1183-1186; Arpinati, et al. (2000) Blood 95:2484-2490; Bolwell, et al. (2003) Bone Marrow Transplant. 31:95-98; Damiani, et al. (2002) Bone Marrow Transpl. 30:261-266; Bauer, et al. (2001) J. Immunol. 166:5000-5007.

Methods for antigen pulsing or loading of DCs, as well as for effecting DC maturation and activation, have been described, see, e.g., Tuettenberg, et al. (2003) Gene Ther. 10:243-250; Guermonprez, et al. (2002) Annu. Rev. Immunol. 20:621-667; Kadowaki, et al. (2001) J. Exp. Med. 194:863-869; Fong and Engelman (2000) Annu. Rev. Immunol. 18:245-273; Shortman and Liu (2002) Nature Revs. Immunol. 21:151-161; Woltman and van Kooten, supra; Motta, et al. (2003) Brit. J. Haematol. 121:240-250; Kadowaki, et al. (2001) J. Exp. Med. 194:863-869; Kadowaki, et al. (2001) J. Immunol. 166:2291-2295. Activation of a DC by stimulating a toll-like receptor (TLR) may be required for the DC to break CD4⁺CD25+T cell-mediated suppression of CD4⁺CD25- T cells, (Pasare and Medzhitov (2003) Science 299:1033-1036).

DCs can be prepared and used for experimental or therapeutic purposes, e.g., for vaccination, see, e.g., Schreurs, et al. (2000) Cancer Res. 60:6995-7001; Panelli, et al. (2000) J. Immunother. 23:487-498; Nestle, et al. (1998) Nature Med. 4:328-332; Bender, et al. (1996) J. Immunol. Methods 196:121-135; Tjoa, et al. (1997) Prostate 32:272-278; Fong and Engleman, supra; Romani, et al. (1994) J. Exp. Med. 180:83-93; Dhodapkar, et al. (1999) J. Clin. Invest. 104:173-180.

V. Purification and Modification of Polypeptides.

Polypeptides, e.g., antigens, antibodies, and antibody fragments, for use in the contemplated method can be purified by methods that are established in the art. Purification may involve homogenization of cells or tissues, immunoprecipitation, chromatography, and use of affinity and epitope tags. Stability during purification or storage can be enhanced, e.g., by anti-protease agents, anti-oxidants, ionic and non-ionic detergents, and solvents, such as glycerol or dimethylsulfoxide.

Modifications of proteins and peptides include epitope tags, fusion proteins, fluorescent or radioactive groups, monosaccharides or oligosaccharides, sulfate or phosphate groups, C-terminal amides, modified N-terminal amino groups, e.g., by acetylation or fatty acylation, intrachain cleaved peptide bonds, and deamidation products (Johnson, et al. (1989) J. Biol. Chem. 264:14262-14271; Young, et al. (2001) J. Biol. Chem. 276:37161-37165). Glycosylation depends upon the nature of the recombinant host organism employed or physiological state (Jefferis (2001) BioPharm 14:19-27; Mimura, et al. (2001) J. Biol. Chem. 276:45539-45547; Axford (1999) Biochim. Biophys. Acta 1:219-229; Malhotra, et al. (1995) Nature Medicine 1:237-243; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391).

VI. Binding Compositions, Agonists, Antagonists, and Muteins.

Human TEASR (hTEASR; a.k.a. 312C2) and mTEASR are described (U.S. Pat. No. 6,111,090 issued to Gorman, et al.; GenBank NM_{—}005092). Anti-TEASR antibodies have been prepared (Shimizu, et al. (2002) Nat. Immunol. 3:135-142; McHugh, et al. (2002) Immunity 16:311-323). Soluble extracellular domains of TEASR-L, soluble extracellular domains of TEASR, and fusion proteins comprising extracellular domains of TEASR and Fc fragments are described (Gurney, et al. (1999) Curr. Biol. 9:215-218; Kwon, et al. (1999) J. Biol. Chem. 274:6056-6061; Shin, et al. (2002) Cytokine 19:187-192; Shin, et al. (2002) FEBS Lett. 514:275-280; U.S. Pat. Pub. No. US 2002/0146389).

Muteins and variants of TEASR-L, TEASR, anti-TEASR-L antibody, and anti-TEASR antibody can be prepared, e.g., by methods involving alanine scanning or mutagenesis of specific residues to any of the 20 classical amino acids, by fusion proteins, by truncations at the N-terminus or C-terminus, or by internal deletions (Shanafelt (2003) Curr. Pharm. Biotechnol. 4:1-20; Park, et al. (1998) J. Biol. Chem. 273:256-261; Leong, et al. (2001) Cytokine 16:106-119; Madhankumar, et al. (2002) J. Biol. Chem. 277:43194-43205; Morrison and Weiss (2001) Curr. Opinion Chemical Biol. 5:302-307). The invention contemplates binding compositions that are agonists, antagonists, or that are neutral, i.e., non-inhibiting and non-stimulating.

Antibodies and binding compositions derived from an antigen-binding site of an antibody are provided. These include human antibodies, humanized antibodies, monoclonal antibodies, polyclonal antibodies, and binding fragments, such as Fab, F(ab)₂, and Fv fragments, and engineered versions thereof. The antibody or binding composition may be agonistic, or antagonistic, or neutral. Antibodies that simultaneously bind to a ligand and receptor are contemplated. Monoclonal antibodies will usually bind with at least a K_{D} of about 1 mM, more usually at least about 300 μM, typically at least about 100 μM, more typically at least about 30 μM, preferably at least about 10 μM, and more preferably at least about 3 μM or better.

Monoclonal, polyclonal, and humanized antibodies can be prepared, see, e.g., Cole, et al. (1985) in Monoclonal Antibodies and Cancer Therapy, Alan R. Liss, Inc., New York, N.Y., pp. 77-96; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378.

A “partially humanized” or “chimeric” antibody contains heavy and light chain variable regions of, e.g., murine origin, joined onto human heavy and light chain constant regions. A “humanized” or “fully humanized” antibody contains the amino acid sequences from the six complementarity-determining regions (CDRs) of the parent antibody, e.g., a mouse antibody, grafted to a human antibody framework. “Human” antibodies are antibodies containing amino acid sequences that are of 100% human origin, where the antibodies may be expressed, e.g., in a human, animal, insect, fungal, plant, bacterial, or viral host (Baca, et al. (1997) J. Biol. Chem. 272:10678-10684; Clark (2000) Immunol. Today 21:397-402).

An alternative to humanization is to use human antibody libraries displayed on phage or human antibody libraries contained in transgenic mice, see, e.g., Vaughan, et al. (1996) Nat. Biotechnol. 14:309-314; Barbas (1995) Nature Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez, et al. (1997) Nature Genet. 15:146-156; Hoogenboom and Chames (2000) Immunol. Today 21:371-377; Barbas, et al. (2001) Phage Display:A Laboratory Manual, Cold Spring Harbor Laboratory Press, Cold Spring Harbor, N.Y.; Kay, et al. (1996) Phage Display of Peptides and Proteins:A Laboratory Manual, Academic Press, San Diego, Calif.; de Bruin, et al. (1999) Nat. Biotechnol. 17:397-399.

Single chain antibodies, single domain antibodies, and bispecific antibodies are described, see, e.g., Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) J. Immunol. 148:1547-1553; U.S. Pat. Nos. 5,932,448; 5,532,210; 6,129,914; 6,133,426; 4,946,778.

Antigen fragments may be joined to other materials, such as fused or covalently joined polypeptides, to be used as immunogens. An antigen and its fragments may be fused or covalently linked to a variety of immunogens, such as keyhole limpet hemocyanin, bovine serum albumin, or ovalbumin (Coligan, et al. (1994) Current Protocols in Immunol., Vol. 2, 9.3-9.4, John Wiley and Sons, New York, N.Y.). Peptides of suitable antigenicity can be selected from the polypeptide target, using an algorithm, see, e.g., Parker, et al. (1986) Biochemistry 25:5425-5432; Jameson and Wolf (1988) Cabios 4:181-186; Hopp and Woods (1983) Mol. Immunol. 20:483-489.

Purification of antigen is not necessary for the generation of antibodies. Immunization can be performed by DNA vector immunization, see, e.g., Wang, et al. (1997) Virology 228:278-284. Alternatively, animals can be immunized with cells bearing the antigen of interest. Splenocytes can then be isolated from the immunized animals, and the splenocytes can fused with a myeloma cell line to produce a hybridoma (Meyaard, et al. (1997) Immunity 7:283-290; Wright, et al. (2000) Immunity 13:233-242; Preston, et al. (1997) Eur. J. Immunol 27:1911-1918). Resultant hybridomas can be screened for production of the desired antibody by functional assays or biological assays, that is, assays not dependent on possession of the purified antigen. Immunization with cells may prove superior for antibody generation than immunization with purified antigen (Kaithamana, et al. (1999) J. Immunol. 163:5157-5164).

Antibody to antigen and ligand to receptor binding properties can be measured, e.g., by surface plasmon resonance (Karlsson, et al. (1991) J. Immunol. Methods 145:229-240; Neri, et al. (1997) Nat. Biotechnol. 15:1271-1275; Jonsson, et al. (1991) Biotechniques 11:620-627) or by competition ELISA (Friguet, et al. (1985) J. Immunol. Methods 77:305-319; Hubble (1997) Immunol. Today 18:305-306). Antibodies can be used for affinity purification to isolate the antibody's target antigen and associated bound proteins, see, e.g., Wilchek, et al. (1984) Meth. Enzymol. 104:3-55.

Antibodies that specifically bind to variants of TEASR-L or to variants of TEASR, where the variant has substantially the same nucleic acid and amino acid sequence as those recited herein, but possessing substitutions that do not substantially affect the functional aspects of the nucleic acid or amino acid sequence, are within the definition of the contemplated methods. Variants with truncations, deletions, additions, and substitutions of regions which do not substantially change the biological functions of these nucleic acids and polypeptides are within the definition of the contemplated methods.

VII. Therapeutic and Diagnostic Uses.

The invention provides methods for the treatment and diagnosis of immune and proliferative disorders, e.g., inflammation and cancer. The invention provides methods for the treatment and diagnosis of immune, inflammatory, and proliferative disorders, including psoriasis and other skin conditions, rheumatoid arthritis, inflammatory bowel disorders (IBD), including Crohn's disease, CD8⁺ T cell mediated disorders, cancer, e.g., leukemia, and tumors. The methods may comprise use of a binding composition specific for a polypeptide or nucleic acid of TEASR or TEASR-L, e.g., an antibody or a nucleic acid probe or primer. Control binding compositions are also provided, e.g., control antibodies, see, e.g., Lacey, et al. (2003) Arthritis Rheum. 48: 103-109; Choy and Panayi (2001) New Engl. J. Med. 344:907-916; Greaves and Weinstein (1995) New Engl. J. Med. 332:581-588; Robert and Kupper (1999) New Engl. J. Med. 341:1817-1828; Lebwohl (2003) Lancet 361:1197-1204. The invention contemplates use of a TEASR agonist to stimulate cell activation or proliferation, e.g., T cell proliferation, e.g., for treating an infection or proliferative condition. Also contemplated is use of a TEASR antagonist to inhibit cell activation or proliferation, e.g., to inhibit T cell proliferation, e.g., for treating an autoimmune or inflammatory condition or for inducing tolerance.

Methods relating to human antigen presenting cells (APCs), including DCs, e.g., for generating large numbers of cells, storage, pulsing of APCs with antigen or with whole cells, administration to a subject, as well as methods for evaluation of response, are described, see, e.g., Panelli, et al. (2000) J. Immunother. 23:487-498; Nestle, et al. (1998) Nature Med. 4:328-332; Steinman and Dhodapkar (2001) Int. J. Cancer 94:459-473; Fong and Engleman (2000) Annu. Rev. Immunol. 18:245-273.

Methods for co-administration or treatment with a second therapeutic agent, e.g., a cytokine, chemotherapeutic agent, antibiotic, or radiation, are well known in the art (Hardman, et al. (eds.) (2001) Goodman and Gilman 's The Pharmacological Basis of Therapeutics, 10^{th} ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., PA). An effective amount of therapeutic will decrease the symptoms typically by at least 10%; usually by at least 20%; preferably at least about 30%; more preferably at least 40%, and most preferably by at least 50%.

Formulations of therapeutic and diagnostic agents may be prepared for storage by mixing with physiologically acceptable carriers, excipients, or stabilizers in the form of, e.g., lyophilized powders, slurries, aqueous solutions or suspensions, see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.;

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone. Guidance for methods of treatment and diagnosis is available (Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The invention also provides a kit comprising a cell and a compartment, a kit comprising a cell and a reagent, a kit comprising a cell and instructions for use or disposal, as well as a kit comprising a cell, compartment, and a reagent.

The broad scope of this invention is best understood with reference to the following examples, which are not intended to limit the inventions to the specific embodiments.

### EXAMPLES

### I. General Methods

Standard methods of biochemistry and molecular biology are described or referenced, see, e.g., in Maniatis, et al. (1982) Molecular Cloning, A Laboratory Manual, Cold Spring Harbor Laboratory, Cold Spring Harbor Press; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.; Innis, et al. (eds.) (1990) PCR Protocols: A Guide to Methods and Applications, Academic Press, N.Y. Standard methods are also found in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4). Methods for producing fusion proteins are described. See, e.g., Invitrogen (2002) Catalogue, Carlsbad, Calif.; Amersham Pharmacia Biotech (2002), Catalogue, Piscataway, N.J.; Liu, et al. (2001) Curr. Protein Pept. Sci. 2:107-121; Graddis, et al. (2002) Curr. Pharm. Biotechnol. 3:285-297.

Methods to sort, identify, and purify cell populations are described, see, e.g., Melamed, et al. (1990) Flow Cytometry and Sorting, Wiley-Liss, Inc., New York, N.Y.; Shapiro (1988) Practical Flow Cytometry, Liss, New York, N.Y.; and Robinson, et al. (1993) Handbook of Flow Cytometry Methods, Wiley-Liss, New York, N.Y. Methods of histology are available, see, e.g., Carson (1997) Histotechnology: A Self-Instructional Text, 2^{nd} ed., Am. Soc. Clin. Pathol. Press, Chicago, Ill.; Bancroft and Gamble (eds.) (2002) Theory and Practice of Histological Techniques, 5^{th} ed., W.B. Saunders Co., Phila., Pa.

Software packages for determining, e.g., antigenic fragments, signal and leader sequences, protein folding, and functional domains, are available, see, e.g., Vector NTI® Suite (Informax, Inc., Bethesda, Md.); GCG Wisconsin Package (Accelrys, Inc., San Diego, Calif.), and DeCypher® (TimeLogic Corp., Crystal Bay, Nev.); Menne, et al. (2000) Bioinformatics 16:741-742. Public sequence databases are also available, e.g., from GenBank and others.

### II. Distribution of TEASR and TEASR-L

Human TEASR is expressed on various human cells, as determined by Taqman® assays (PE Applied Biosystems, Foster City, Calif.), where results are relative to ubiquitin expression (Table 1). Ubiquitin expression is set to one. (−) means<1; (+) means 1-10; (++) means 10-100; (+++) means 100-500; (++++) means 500-1000; (+++++) means 1000-5000; (++++++) means 5000-10000; (+++++++) means 20,000-30,000; relative to ubiquitin expression of 1.0.

FACS analysis was also used to determine expression. TEASR is highly expressed on CD25⁺CD4⁺ T cells, with little or no expression on CD25⁻CD4⁺ T cells, as determined by FACS analysis of fresh human peripheral blood mononuclear cells (PBMC) separated into pure CD25⁺CD4⁺ T cells and pure CD25⁻CD4⁺ T cells (Table 1). FACS analysis also demonstrated that the CD25⁺CD4⁺ T cells were CD69 negative, HLA-DR low, CD45RO high, and CD45Ra moderate, whereas the CD25⁻CD4⁺ T cells were CD69 negative, HLA-DR negative, CD45RO moderate, and CD45RA high. TEASR expression was monitored with anti-TEASR antibody (27H3D3) and the isotype control. Phenotypes were analyzed with the appropriate antibody and the isotype control antibody (Table 1). CD40L was supplied as CD40-Lc. “CD40L-Lc” is an L cell expressing human CD40L (Denepoux, et al. (2000) J. Immunol. 164:1306-1313).

The time course for TEASR expression on T cells was studied following cell activation (Table 1). Human naïve CD4⁺ T cells and human naïve CD8⁺ T cells were treated with anti-CD3 antibody followed by analysis of TEASR expression. Treatment was for 0, 6, 12 (not shown), 24, 48 (not shown), or 72 h, where analysis was by FACS analysis. At 0 and 6 h, CD4⁺ T cells and CD8⁺ T cells showed no detectable expression, while progressively increases in expression occurred at 12-48 h, while nearly 100% of the cells showed expression at 72 h (Table 1).

TABLE 1

| Expression of TEASR by | TEASR expression by |
|---|---|
| human cells and tissues | Taqman ®analysis |
| PreDC2 fresh | +++ |
| PreDC2, IL-3 (3 days) | +++++ |
| PreDC2, IL-3 (3 days) CD40L-Lc (3 days) | ++++++ |
| PreDC2, HSV, 3 days | +++++ |
| Monocyte, GMCSF (5 days) IL-4 (5 days) | +++++ |
| CD40-Lc (24 h) | |
| Monocyte GMCSF (5 days) IL-4 (5 days) | +++++ |
| Macrophages (monocytes + M-CSF) | +++++++ |
| Human monocyte/PBMC resting | +++ |
| Human monocyte/PBMC activated LPS | +++++ |
| Human monocyte/PBMC aCD3/aCD28 | ++++++ |
| activated | |
| Human Th1 cell resting | +++++ |
| Human Th1 cell aCD3/aCD28 activated | ++++++ |
| Human Th2 cell resting | ++++ |
| Human Th2 cell aCD3/TPA activated | ++++++ |
| B/T cell splenocytes resting | +++ |
| B/T cell splenocytes activated aCD40 + IL-4 | +++++ |
| Neutrophil untreated | + |
| Neutrophil activated PMA | ++ |
| Human NK cell resting | ++++ |
| Human NK cell PMA/ionomycin activated | +++++ |
| Human dendritic cell resting | + |
| Human dendritic cell activated LPS | ++++ |
| Human dendritic cell pre-DC2, leukemia | +++++ |
| Human skin control | (−) |
| Human skin psoriasis | +++ |
| Human synovia ischemic heart disease | ++ |
| Human synovia rheumatoid arthritis | +++ |
| | TEASR expression by |
| | FACS analysis |
| CD25⁺ CD4⁺ T cells from PBMC. | Positive |
| CD25⁻ CD4⁺ T cells from PBMC. | Negative |
| CD4⁺ PBMC phorbol myristate acetate, | Positive |
| ionomycin, 48 h | |

Time course of TEASR expression by CD4⁺ T cells after activation

| | |
|---|---|
| Human naïve CD4⁺ T cells, no anti-CD3. | Negative |
| Human naïve CD4⁺ T cells, anti-CD3, 6 h. | Negative |
| Human naïve CD4⁺ T cells, anti-CD3, 24 h. | Positive for ˜50% of cells |
| Human naïve CD4⁺ T cells, anti-CD3, 72 h. | Positive for ˜95% of cells |

Time course of TEASR expression by CD8⁺ T cells after activation

| | |
|---|---|
| Human naïve CD8⁺ T cells, no anti-CD3. | Negative |
| Human naïve CD8⁺ T cells, anti-CD3, 6 h. | Negative |
| Human naïve CD8⁺ T cells, anti-CD3, 24 h. | Positive for ˜50% of cells |
| Human naïve CD8⁺ T cells, anti-CD3, 72 h. | Positive for ˜95% of cells |

Human TEASR ligand (a.k.a. TEASR-L) expression was measured on various human cells and tissues (Table 2). (−) means <1; (+) means 1-10; (++) means 10-100; (+++) means 100-500; (++++) means 500-1000; (+++++) means 1000-5000; (++++++) means 5000-10000, relative to ubiquitin expression of 1.0. ND means not determined. CD40L was supplied as CD40-Lc. Freshly isolated preDC2 express relatively little TEASR-L, where expression can be induced by treatment with IL-3 alone (3 days), or by IL-3 (3 days) and CD40L (24 h) (Table 2). The FACS data indicate the signal with anti-TEASR-L antibody relative to that with isotype control antibody.

TABLE 2

| Expression of TEASR-L (stud #1) | Taqman ® analysis | |
|---|---|---|
| Human T cell TH0 resting | (−) | |
| Human T cell TH0 activated | ++ | |
| aCD3/aCD28 | | |
| Human NK cell resting | (−) | |
| Human NK cell IL-2 activated | ++ | |
| Human DC resting | ++ | |
| Human DC activated | ++++ | |
| PMA/ionomycin | | |
| Human skin control | + | |
| Human skin psoriasis | ++ | |
| Human colon control | + | |
| Human colon Crohn's | ++ | |
| Expression of TEASR-L (study #2) | Taqman ® analysis | FACS analysis |
| Fresh preDC2 | (−) | (−) |
| PreDC2 + IL-3 (3 days) | +++++ | +++ |
| PreDC2 + IL-3 (3 days) + | ND | +++ |
| CD40L (24 h) | | |
| PreDC2 + IL-3 (3 days) + | (−) | + |
| CD40L (3 days) | | |
| PreDC2 + HSV (3 days) | +++++ | ND |
| PreDC2 +CpG (3 days) | ND | +++ |
| PreCD2 + CpG (3 days) + | ND | +++ |
| CD40L (24 h) | | |
| Monocyte, GMCSF (5 days) IL-4 | ++++ | ND |
| (5 days) CD40-Lc (24 h) | | |
| Monocyte GMCSF (5 days) IL-4 | (−) | ND |
| (5 days) | | |
| Macrophages | +++ | ND |
| (monocytes + M-CSF) | | |

### III. Assay Method for Functional TEASR-L

Ba/F3 cells were transfected with a fusion protein comprising the extracellular domain of hTEASR and the cytoplasmic region of Fas. Stimulation of the expressed TEASR fusion protein resulted in cell death, allowing measurement of direct stimulation of TEASR by anti-TEASR antibody. Apoptotic cell death, used as a measure of TEASR activity, was assessed by measuring ⁵¹Cr-chromium release from ⁵¹Cr-labeled Ba/F3 cells.

Transfected Ba/F3 cells were exposed to IL-3-stimulated DC2 cells, and monitored for apoptotic cell death. L-3-treated DC2 provoked apoptotic cell death of the transfected Ba/F3 cells (about 23% release of ⁵¹Cr), in the presence of control IgG1, demonstrating that IL-3-stimulated DC2 expressed TEASR-L and can transmit a signal to a TEASR-transfected cell. With anti-TEASR-L antibody (11A7.2D9), cell death was minimal (about 8% release), demonstrating that signaling was specifically dependent on TEASR-L to TEASR signaling.

Control experiments using non-transfected Ba/F3 cells demonstrated that exposing these cells to IL-3-treated DC2 resulted in minimal increases in ⁵¹Cr-release. Here release was only 4% in the presence of control IgG1 and only 8% in the presence of anti-TEASR-L antibody (11A7.2D9). Release in the range of, e.g., 4-8%, is believed to reflect spontaneous cell death and ⁵¹Cr-leakage, and not apoptotic cell death.

### Example IV

### DC2 Breaks the Suppressive Activity of CD25⁺CD4⁺ T Cells

Treg cell-mediated suppression of activated T cells was demonstrated in a first study, followed by a second study demonstrating DC2-mediated abrogation of the above-described Treg cell-mediated suppression of naïve CD4⁺ T cells.

CD25⁺CD4⁺ Treg cell-mediated suppression of activated naïve CD4⁺ T cells was demonstrated (first study). In this particular example, the naïve CD4⁺ T cells were activated by DC1 cells. DC1-mediated stimulation of CD4⁺ T cell proliferation in absence of Treg cells was shown by an increase in ³H-thymidine uptake of about 26,000 cpm, which corresponds to maximal proliferation in this example. Separate cell incubation mixtures were titrated with different amounts of regulatory CD25⁺CD4⁺ Treg cells, i.e., at ratios of CD25⁺CD4⁺ Treg cells/naïve CD4⁺ T cells of 0/8, 1/8, 2/8, 4/8, and 1/1, with constant levels of DC1 cells. CD4⁺ T cell proliferation was inhibited, where the 1:1 ratio resulted in the maximal detected inhibition, i.e., under 20% maximal proliferation of the naïve CD4⁺ T cells. Tritium uptake in the presence of the Treg cells and DC1s only (no naïve CD4⁺ T cells) was about 1000 cpm or less, demonstrating that ³H-thymidine uptake reflects proliferation of the naïve CD4⁺ T cells. Thus, CD25⁺CD4⁺ Treg cells can inhibit or suppress DC-dependent proliferation of naïve CD4⁺ T cells.

DC2-dependent abrogation of CD25⁺CD4⁺ Treg-mediated suppression of activated naïve CD4⁺ T cell proliferation was demonstrated (second study). Three different preparations of DC2 cells were tested for their ability to break or abrogate CD25⁺CD4⁺ Treg-mediated suppression of the CD4⁺ T cells. In each case, the DC2 preparation also served to directly stimulate the CD4⁺ T cells.

The three preparations of DC2s were, Preparation #1: IL-3-treated (6 days) pre-DC2 cells; Preparation #2: IL-3+CD40L-treated (6 days simultaneous treatment with both IL-3 and CD40L) pre-DC2 cells, and Preparation #3: IL-3 (6 days total)+CD40L (last 24 h of the 6 days)-treated pre-DC2 cells. The source of CD40L was L cells transfected with CD40L. IL-3 was used at 10 ng/ml (R & D Systems, Inc., Minneapolis, Minn.). CD40L-Lc cells were used at a concentration of 10000 to 50000 L cells/well of a 96 well flat bottom plate.

The above three preparations of DCs were shown to stimulate directly proliferation of naïve CD4⁺ T cells. Direct stimulation of the DC2s to naïve CD4⁺ T cells resulted in T cell proliferation, where Preparation #3 resulted in the highest level of proliferation (Table 3).

TABLE 3
Stimulation of naïve CD4⁺ T cells by three DC2 cell preparations.

| Preparation | | Proliferation, ³H-thymidine uptake. |
|---|---|---|
| #1. | IL-3 for 6 days. | 18,000 cpm |
| #2. | IL-3 + CD40L for 6 days. | 35,000 cpm |
| #3. | IL-3 for 5 days, followed by | 55,000 cpm |
| | IL-3 + CD40L for 24 hours. | |

In a separate study, the incubation mixtures described in Table 3 were supplemented with CD25⁺CD4⁺ Treg cells, where the resulting data on naïve CD4⁺ T cell proliferation are shown in Table 4. The CD25⁺CD4⁺ Treg cells suppressed proliferation of the naïve CD4⁺ T cells, in mixtures supplemented with Preparations #1 or #2 DC2s (Table 4). Here, proliferation was low, i.e., tritium uptake was only about 300 cpm. However, proliferation was near maximal with supplementation by Preparation #3 DC2s (Table 4), demonstrating that this preparation of DC2s break Treg cell-mediated suppression of T cells. Here, tritium uptake was about 50,000.cpm.

Separate control studies demonstrated that incubation mixtures containing regulatory CD25⁺CD4⁺ Treg cells and DC2s, but without naïve CD4⁺ T cells, took up little tritium, i.e., only about 100 cpm. Cell proliferation studies using the 5-(and-6)-carboxyfluorescein diacetate succinimidyl ester (CFSE) dilution method and the IL-2Ralpha chain expression method, measures of cell proliferation, confirmed the ³H-thymidine incorporation results of Table 4.

Methodology for measuring cell proliferation was as follows. Staining with CFSE, followed by cell division results and dilution of CFSE, is used as a measure of proliferation. CFSE (Molecular Probes, Inc., Eugene, Oreg.) is a fluorescent dye used to monitor cells without interfering with viability (Dumitriu, et al. (2001) Analyt. Biochem. 299:247-252; Sheehy, et al. (2001) J. Immunol. Methods 249:99-110). IL-2Ralpha chain is a key regulator of lymphocyte proliferation, and its expression is used as a proliferation marker (Eicher, et al. (2002) Cytokine 17:82-90; Kim and Leonard (2002) EMBO J. 21:3051-3059).

TABLE 4
Naïve CD4⁺ T cell proliferation as determined by ³H-thymidine uptake.
CELLS IN INCUBATION MIXTURES

| Preparation of DC2 cells | | | CD25⁺ CD4⁺ | | |
|---|---|---|---|---|---|
| #1 | #2 | #3 | Treg cells | Naïve CD4⁺ cells | Proliferation |
| Yes | — | — | Yes | Yes | Low |
| — | Yes | — | Yes | Yes | Low |
| — | — | Yes | Yes | Yes | High |

Unlike DC2 cells, DC1 cells do not to abrogate the suppressive function of CD25⁺CD4⁺ T cells. Immature DC1s were prepared by incubating CD4⁺CD11c+CD14+monocytes with GM-CSF and IL-4 for six days. The immature DC1 s were subsequently treated for 24 h with: (1) CD40L to provide mature DC1s; (2) Lipopolysaccharide (LPS) to provide mature DC1; (3) CD40L and LPS to provide mature DC1 s; or (4) Medium only. Proliferation of naïve CD4⁺ T cells was assessed by ³H-thymidine uptake. Naïve CD4⁺ T cells were incubated with each of the preparations of DC1 and in each case high proliferation was found, i.e., 47,000 cpm, 44,000 cpm, 35,000 cpm, and 43,000 cpm for the four respective mixtures of DC1 cells and naïve CD4⁺ cells. Supplementation of each of the above four mixtures with regulatory CD25⁺CD4⁺ T cells in all cases suppressed CD4⁺ T cell proliferation, i.e., resulting in tritium uptake of 10,000 to 13,000 cpm. Thus DC1 cells do not abrogate or break the suppressive effects of CD25⁺CD4⁺ T cells on naïve CD4⁺ T cell proliferation.

Alternate embodiments of the mature DC2s to Preparation #3 (Table 4) are provided. The invention contemplates a total period of exposure to IL-3 (first interval) of, e.g., 2, 3, 4, 5, 6, 7, or 8 days, or more, and the like, or to any interval comprising a fractional period of a day. The invention contemplates a total period of exposure to a CD40L agonist (second interval) of 6 h, 12 h, 18 h, 24 h, 36 h, 48 h, or 72 h, or more, or 1 to 72 h or longer, or the like, or any interval comprising a fractional period of an hour. The method can also be modified by changing the relative positions of the first and second intervals, e.g., where the second interval occurs immediately after the first interval, occurs immediately prior to the end of the first interval, or where the second interval is centered in the first interval, and the like. Treatment involving a first reagent for a first period of days of six days (days 1-6) and treatment with a second reagent for a second period of days of one day (day 6), means that the second reagent is not added or introduced until about the end of day 5 or until about the beginning of day 6. Modifications can also comprise interruptions, e.g., for the washing, storage, cooling, or freezing of cells. These modifications can be made and tested by routine screening. Routine screening can involve, e.g., assessing the ability of the mature DC2s (equivalent to Preparation #3) to break Treg-mediated suppression of T cell proliferation to a greater extend than mature DC2s prepared, e.g., by exposure to IL-3 alone, or the ability of the mature DC2s (equivalent to Preparation #3) to stimulate T cell proliferation to a greater extent than mature DC2s prepared, e.g., by exposure to IL-3 alone.

### Example V

### TEASR Agonists Stimulate T Cell Proliferation

Anti-TEASR antibody stimulated proliferation of human CD8⁺ T cells (Table 5, mixture #3) but not of human CD4⁺ T cells (Table 5, mixture #1). In these studies, the antibody was presented to the T cells in the form of a complex with CD32 L cells (feeder cells). Table 5 also reveals some dependence on anti-CD3 concentration for the stimulatory effect.

CD32/CD58/CD80 L cells were also used as feeder cells. Here, anti-TEASR antibody enhanced proliferation of anti-CD3-stimulated CD4⁺ T cells (Table 5, mixture #2) as well as of anti-CD3-stimulated CD8⁺ T cells (Table 5, mixture #4). Here, CD58 and CD80 serve as co-stimulatory agents to the T cells. Again, Table 5 shows some dependence on anti-CD3 concentration for the stimulatory effect.

Anti-TEASR antibody was compared with hTEASR-L-Ig fusion protein for their ability to stimulate T cell proliferation. These two TEASR agonists were compared in their ability to stimulate CD4⁺ T cells in the presence of CD32/CD58/CD80 L cells, and to stimulate CD8⁺ T cells in presence of CD32 L cells. Anti-TEASR antibody increased proliferation of CD8⁺ T cells in the presence of CD32 feeder L cells by 3.7-fold, while the fusion protein increased proliferation by about 5.6-fold. Anti-TEASR antibody increased proliferation of CD4+ T cells in the presence of CD32/CD58/CD80L feeder L cells by about 1.6-fold, while the fusion protein increased proliferation by about 2.5-fold. All studies utilizing hTEASR-L-Ig fusion protein utilized control incubations with rat IgG2a (25 μg/ml), human IgG (25 μg/ml), or no added antibody.

TABLE 5
Anti-TEASR antibody-mediated T cell proliferation.
Components of cell mixtures.

| Cell mixture #1 | Cell mixture #2 | Cell mixture #3 | Cell mixture #4 |
|---|---|---|---|
| CD32 L cells | CD32/CD58/ | CD32 L cells | CD32/CD58/ |
| | CD80 L cells | | CD80 L cells |

| | |
|---|---|
| CD4⁺ T cells | CD8⁺ T cells |

Anti-CD3 antibody (10⁻⁶ to 10 μg/ml)
Anti-TEASR antibody (25 μg/ml)
Fold-stimulation of proliferation by anti-TEASR antibody

| | | | |
|---|---|---|---|
| No increase. | 30-50% increase at | 50% increase at | 2-4-fold increase |
| | 10⁻⁶ to 10⁻⁵ μg/ml | at about 10⁻² to | at about 10⁻⁴ to |
| | anit-CD3. | 10 μg/ml | 10⁻¹ μg/ml |
| | | anti-CD3. | anti-CD3. |

The conditions for cell activation were as follows. Irradiated CD32 L cells (feeder cells) or irradiated CD32/CD58/CD80 L cells (feeder cells) were incubated for 2 h, followed by addition of anti-CD3 antibody (Spv-T3b) and anti-TEASR agonistic antibody (3D6.A2). Anti-CD3 antibody was used at titrating concentrations from 10⁻⁶ to about 10² micrograms/ml. After addition of antibodies, cells were incubated 1 h, and then purified human CD4⁺ naïve T cells or CD8+ naïve T cells, obtained from the same human donor, were introduced to provide completed cell mixtures. Completed cell mixtures were then incubated 5 days, followed by assessment of proliferation by ³H-thymidine uptake or by flow cytometry.

The feeder cells served as a source of CD32, or of CD32, CD58, and CD80, for use in signaling to the T cell. CD32 (a.k.a. FcγRII), an Fc receptor, served to fix the added antibodies or fusion protein on the surface of the L cell for presentation, e.g., to the naïve CD8⁺ T cell. CD58 is used for adhesion and/or to transmit a signal to its ligand, CD2 (Zaru, et al. (2002) J. Immunol. 168:4287-4291). The L cells and conditions for transfection are described, see, e.g., Sornasse, et al. (1996) J. Exp. Med. 184:473-483; Demeure, et al. (1994) J. Immunol. 152:4775-4782; McRae, et al. (1998) J. Immunol. 160:4298-4304; Lanier, et al. (1995) J. Immunol. 154:97-105; Azuma, et al. (1993) J. Immunol. 150:1147-1159; Azuma, et al. (1992) J. Exp. Med. 175:353-360; Azuma, et al. (1992) J. Immunol. 149:1115-1123.

### Example VI

### Cell Preparation

Human plasmacytoid cells were prepared as follows. Plasmacytoid pre-DCs were isolated from peripheral blood of healthy donors by Ficoll-Hypaque centrifugation (Amersham Pharmacia Biotech, Piscataway, N.J.). T, B, NK cells, monocytes, and erythrocytes were depleted from blood mononuclear cells by using mouse anti-CD3 (OKT-3), anti-CD14 (RPA-M1), anti-CD19 (Leu-12), anti-CD56 (Leu-19), anti-glycophorin A (10F7MN) mAbs, and magnetic beads coated with goat anti-mouse IgG (Dynabeads® M-450) (Dynal, Inc., Lake Success, N.Y.). The resulting cells were stained with Tri-color®-conjugated anti-CD4 (Caltag Laboratories, Inc., Burlingame, Calif.), phycoerythrin (PE)-conjugated anti-CD11c (BD Pharmingen, San Diego, Calif.), and a cocktail of FITC-conjugated anti-CD3, anti-CD14, anti-CD16, and anti-CD20 mAbs (BD Pharmingen). CD4⁺CD11c⁻CD3⁻CD14⁻CD16⁻CD20⁻ cells were isolated by cell sorting as plasmacytoid pre-DC (purity >99%).

CD4⁺ and CD8⁺ T cells were isolated from adult human blood as follows. Naïve CD4⁺ T cells were enriched from peripheral blood mononuclear cells by immunomagnetic deletion using mouse anti-CD8, anti-CD14, anti-CD16, anti-CD19, anti-HLA-DR, and anti-CD45RO mAb, followed by magnetic beads coated with goat anti-mouse IgG. These cells were stained by Tri-color®-conjugated anti-CD4 mAb (Caltag, Inc.), and a cocktail of fluorescein isothiocyanate (FITC)-conjugated anti-CD8, anti-TCR-γδ, anti-CD14, anti-CD16, anti-CD20, and anti-CD25 mAbs (BD PharMingen). CD4⁺lineage⁻ cells were isolated by fluorescence-activated cell sorting and were >98% CD4⁺ T cells. Naïve CD8⁺ T cells were enriched from peripheral blood mononuclear cells by immunomagnetic deletion using mouse anti-CD4, anti-CD14, anti-CD56, anti-CD19, anti-HLA-DR, and anti-CD45RO mAb, followed by magnetic beads coated with goat anti-mouse IgG. These cells were stained with APC-conjugated anti-CD8, PE-Cy5-conjugated anti-CD45RA, PE-conjugated CD27, and a cocktail of FITC-conjugated anti-CD4, anti-TCRγδ, anti-CD14, anti-CD16, and anti-CD20 mAbs (BD Pharmingen). CD8⁺CD27⁺CD45RA⁺lineage cells were isolated by fluorescence-activated cell sorting and were >98% CD8⁺ T cells. CD8⁺CD27⁺CD45RA⁺ have been previously described as naïve CD8⁺ T cells.

Cell proliferation was stimulated and assessed as follows. Irradiated transfectant L cells (1×10⁴ cells /well) were plated and incubated for 2 h in 96-well U-bottom microtiter plate in Yssel's Medium (Gemini Bio-Products, Woodland, Calif.) supplemented with 10% fetal bovine serum. Then, anti-CD3 (clone Spv-T3b) and, anti-TEASR mAb (clone 3D6.A2) or rat IgG2a isotype control (R&D System, Minneapolis, Minn.) were added to the each well at the concentration indicated in figures. After incubation for 2h, purified T cells were added at 1-2×10⁴ cells /well. The culture was incubated for 96-h and during the last 12-h of culture, 1 μCi of ³H-thymidine (DuPont NEN, Boston, Mass.) was added to each well and cellular incorporation was determined.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of modulating proliferation of a human cell comprising contacting the cell with:
a) an agonist of glucocorticoid-induced tumor necrosis factor family-related receptor (TEASR) or of TEASR-L ligand (TEASR-L); or
b) an antagonist of TEASR or of TEASR-L.

2. The method of claim 1, wherein the agonist increases cell proliferation

3. The method of claim 1, wherein the antagonist decreases cell proliferation.

4. The method of claim 1, wherein the cell is a human CD8⁺ T cell.

5. The method of claim 1, wherein the agonist or antagonist is a binding composition that specifically binds to TEASR or to TEASR-L.

6. The method of claim 5, wherein the binding composition is derived from the antigen binding site of:
a) an anti-TEASR antibody; or
b) an anti-TEASR-L antibody.

7. The method of claim 5, wherein the binding composition is:
a) a polyclonal antibody;
b) a monoclonal antibody;
c) a human antibody or a humanized antibody;
d) an Fab or F(ab′)₂ fragment;
e) a peptide mimetic of an antibody;
f) a soluble TEASR or soluble TEASR-L; or
g) detectably labeled.

8. A method of treating a human immune disorder comprising treatment or administration with an antagonist of TEASR.

9. The method of claim 8, wherein the immune disorder is:
a) psoriasis;
b) rheumatoid arthritis;
c) an inflammatory bowel disorder (IBD); or
d) a CD8⁺ T cell-mediated disorder.

10. The method of claim 8, wherein the antagonist of TEASR is a binding composition that specifically binds to TEASR-L.

11. The method of claim 10, wherein the binding composition is:
a) a polyclonal antibody;
b) a monoclonal antibody;
c) a human antibody or a humanized antibody;
d) an Fab or F(ab′)₂ fragment;
e) a peptide mimetic of an antibody;
f) a soluble TEASR; or
g) detectably labeled.

12. A method of treating a human proliferative disorder comprising treatment or administration with an agonist of TEASR.

13. The method of claim 12, wherein the agonist comprises a binding composition that specifically binds to TEASR.

14. The method of claim 13, wherein the binding composition is:
a) a polyclonal antibody;
b) a monoclonal antibody;
c) a human antibody or a humanized antibody;
d) an Fab or F(ab′)₂ fragment;
e) a peptide mimetic of an antibody;
f) a soluble TEASR-L; or
g) detectably labeled.
